(19) United States
(12) Patent Application Publication
(10) Pub. No.: US 2007/0119752 A1
(43) Pub. Date: May 31, 2007

(54) Method for removing hydrocarbons from a vapor-gas medium formed during petroleum product storage and when filling a tank therewith

(75) Inventors: Valery Grigorievich Tsegelsky, Moscow (RU); Alexandr Nikolaevich Reutov, Moscow (RU)

Correspondence Address: JACOBSON HOLMAN PLLC, 400 SEVENTH STREET N.W., SUITE 600, WASHINGTON, DC, 20004, US

(73) Assignee: VALERY GRIGORIEVICH TSEGELSKY, Moscow (RU)

(21) Appl. No.: 10/561,523

(22) PCT Filed: Mar. 19, 2004
(86) PCT No.: PCT/RU04/00107

§ 371 Date: Dec. 20, 2005

(30) Foreign Application Priority Data
Jun. 20, 2003 (RU) 2003118273
Sep. 12, 2003 (RU) 2003127687

(51) Int. Cl. C10G 25/03; C07C 7/12; C07C 7/13

(52) U.S. Cl. 208/310 R

(57) ABSTRACT

The invention relates to petrochemistry, specifically to removing hydrocarbons from a vapour-gas medium formed during petroleum product storage. The inventive method consists in pump supplying a liquid medium to a liquid/gas jet device, pumping out said vapour-gas medium from a container filled with a petroleum product or from a petroleum product storage tank, compressing the vapour-gas mixture in the liquid/gas jet device by a liquid-medium energy. A gaseous phase is lead from a separator to an absorption column, wherein the absorption of hydrocarbons from the gaseous phase is carried out by means of a hydrocarbon liquid which is supplied to said column in the form of an absorber. The hydrocarbon-free gaseous phase and the hydrocarbon liquid containing hydrocarbons dissolved therein are separately evacuated from the absorption column. Gasoline or kerosene are used in the form of a petroleum product and a hydrocarbon liquid and cooled to a temperature ranging from minus 10° C. to minus 50° C. prior to the supply thereof to the absorption column, and the pressure of the vapour-gas and liquid media mixture which is formed in the liquid/gas jet device is maintained in the separator at a level ranging from 0.2 MPa to 1.5 MPa. Said invention makes it possible to increase the efficiency of hydrocarbon removal from the vapour-gas medium with low electric energy consumption.

### FIELD OF THE INVENTION

The present invention relates to methods using a pump-ejection installation in systems for removing hydrocarbons exhausted into atmosphere from a vapor-gas medium formed during petroleum product storage and when filling a tank therewith.

### BACKGROUND OF THE INVENTION

A method is known for storing and filling evaporated products, comprising feeding liquid products with a pump into a container and removing therefrom the vapors of the product fed therein (RU 2035365, B 65 D 90/30, 20 May 1995).

This method of storing and filling provides for the removal of vapors of a liquid product from a container, but this method is relatively complex, since it requires, in addition to the use of a system for condensing vapors in a refrigerator with removal of the condensate into a special tank, the use of a system for removal of non-condensed vapors and gases (including air) into a tank from which the evaporating product is fed into a container.

The method most similar to the invention with respect to technical essence and achieved technical result is the method for removing hydrocarbons from a vapor-gas medium formed during storage of petroleum products and when filling a tank therewith, comprising feeding a liquid medium with a pump into a liquid-gas jet device, pumping a vapor-gas medium by the latter from a tank filled with petroleum products or from a storage tank for storage of petroleum products and compressing it in the liquid-gas jet device as a result of the energy of the liquid medium, feeding a mixture of vapor-gas and liquid mediums formed in the liquid-gas jet device into a separator, separating the mixture in the separator to a gaseous phase and a liquid medium with removal of the gaseous phase and the liquid medium from the separator, wherein the gaseous phase from the separator is directed to an absorption column into which a hydrocarbon liquid is fed as the absorbent, the process of absorption of hydrocarbons from the gaseous phase by the hydrocarbon liquid is carried out in the absorption column, after which the hydrocarbon-removed gaseous phase and the hydrocarbon liquid with the hydrocarbons of the gaseous phase dissolved therein are separately removed from the absorption column (RU 2193443, B 65 D 90/30, 27 Nov. 2002).

This method provides for compression and condensation of hydrocarbon vapors of petroleum products, reduces the concentration of harmful for the environment hydrocarbon vapors in the exhausted into atmosphere vapor-gas medium formed during the storage of petroleum products or filling a tank therewith. However, when hydrocarbon vapors of relatively volatile petroleum fractions, for example, gasoline or kerosene, are pumped out, the effectiveness of this method for removing hydrocarbons from a vapor-gas medium is reduced.

### SUMMARY OF THE INVENTION

The object of the instant invention is to reduce the loss of petroleum products and increase the effectiveness of removing hydrocarbons and other organic compounds of the exhausted into atmosphere a vapor-gas medium formed during the storage of petroleum products and when filling a tank therewith.

The stated object is achieved in that a method for removing hydrocarbons from a vapor-gas medium formed during storage of petroleum products and when filling a tank therewith comprises pump supplying a liquid medium to a liquid-gas jet device, pumping out with this device the vapor-gas medium from a tank filled with petroleum products or from a petroleum product storage container, compressing the vapor-gas medium in the liquid-gas jet device by energy of the liquid medium, feeding a mixture of the vapor-gas and liquid mediums formed in the liquid-gas jet device to a separator, separating the mixture in the separator into a gaseous phase and a liquid medium with removal of the gaseous phase and the liquid medium from the separator, wherein the gaseous phase is fed from the separator to an absorption column, into which a hydrocarbon liquid is fed as an absorbent, a process of absorption of hydrocarbons from the gaseous phase is carried out by the hydrocarbon liquid in the absorption column, then the hydrocarbon-removed gaseous phase and the hydrocarbon liquid containing hydrocarbons of the gaseous phase dissolved therein are separately evacuated from the absorption column, wherein a gasoline or kerosene fraction of petroleum refining is used as the hydrocarbon liquid, and prior to being fed to the absorption column the hydrocarbon liquid is cooled to a temperature within the range of from minus 10° C. to minus 50° C., and the pressure of the mixture of the vapor-gas and liquid mediums, which is formed in the liquid-gas jet device is maintained in the separator at a level ranging from 0.2 to 1.5 MPa.

Furthermore, a portion of the liquid medium from the separator may be fed to a pump input, and the gaseous phase after exit from the absorption column may be additionally cooled, wherein a condensate formed as a result of cooling the gaseous phase is separated therefrom in an additional separator, and the gaseous phase is fed from the additional separator to a vortical pipe in which the gaseous phase is separated into cold and warm gas mediums, then the warm gas medium is removed into the atmosphere and the cold gas medium is used to cool the gaseous phase after the exit of the latter from the absorption column.

The gaseous phase from the absorption column may be fed to a gasdynamic separator in which the gaseous phase as a result of its acceleration and expansion is cooled with the formation in the stream of a condensate from the hydrocarbon vapors remaining in the gaseous phase, then the condensate is separated from the gaseous phase and removed from the gasdynamic separator.

The hydrocarbon liquid with the hydrocarbons of the gaseous phase dissolved therein may be fed from the absorption column to a separator or to a pump input.

The hydrocarbon liquid with the hydrocarbons of the gaseous phase dissolved therein may be fed from the absorption column into a container for storing petroleum products or a tank to be filled therewith.

The liquid medium may be removed from the separator into a container for storing petroleum products or a tank to be filled therewith.

Petroleum products, in particular gasoline or kerosene fractions of petroleum refining may be fed to the separator or pump input.

The gaseous phase after exit from the absorption column may be fed to a membrane device in which the gaseous hydrocarbons remaining therein are separated, then the gas leaned of hydrocarbons and the hydrocarbon-enriched gas are separately removed from the membrane device.

The hydrocarbon-enriched gas may be pumped from the membrane device by the liquid-gas jet device.

The hydrocarbon-enriched gas may be pumped from the membrane device by an additional liquid-gas jet device.

The vapor-gas medium, which is formed in containers for the storage of petroleum products and also in the process of filling—evacuating different types of tanks, consists mainly of air and hydrocarbon vapors. For example, in the case of gasoline, the content of vapors of petroleum products in a vapor-gas medium may vary within the range of from 300 to 1500 g/m³ and more. Such a significant content of hydrocarbons in the vapor-gas medium when it is exhausted into the atmosphere results in both contamination of the environment and in the loss of a significant amount of a marketable petroleum product, in this case gasoline. Therefore the removal of hydrocarbons from the vapor-gas medium is an actual object. It is necessary to prevent the ingress of hydrocarbons into the atmosphere and reduce the loss of a marketable petroleum product.

The claimed method makes it possible to reduce the concentration of hydrocarbons in a vapor-gas medium to a concentration below the maximum permissible level of vapor exhaust into the environment by pumping out and compressing the vapor-gas medium with the aid of a pump-ejection installation and then removing the hydrocarbons from the medium. Wherein it is most advisable to simultaneously use the liquid medium, which is fed by a pump into the liquid-gas jet device as the ejection medium, to both pump out the vapor-gas medium from the tank to be filled with petroleum products or from the container for storage of petroleum products and to absorb hydrocarbons harmful for the environment from the vapor-gas medium being pumped out. Petroleum products stored in the container or filling the tank, in particular gasoline or kerosene fractions of petroleum refining, may be used as such a liquid medium. However, in order to provide for an effective process of absorption of hydrocarbons from the vapor-gas medium, conditions should be created under which the hydrocarbon liquid used as the absorbent would have the pressure of saturated vapors at a temperature of feeding it into the absorption column, preferably significantly lower than the pressure of saturated vapors of the hydrocarbons in the vapor-gas medium formed during the storage and transfer of the petroleum products. Furthermore, it is necessary to achieve a significant reduction of the evaporation of the hydrocarbon liquid itself in the process of absorption. It was established during research that most advisable from the economical point of view is a reduction of the temperature of the hydrocarbon liquid (gasoline, kerosene) to a temperature, which is within the range of from minus 10° C. to minus 50° C. prior to feeding it into the absorption column. At a temperature of the hydrocarbon liquid above minus 10° C., the required effectiveness of absorption of hydrocarbons from the vapor-gas medium is not achieved, and at a temperature below minus 50° C. there is a significant increase of the consumption of electric energy by the refrigerating machine, which is not compensated by an increase of the absorption capability of the hydrocarbon liquid. Simultaneously with a reduction of the temperature, the viscosity of the hydrocarbon liquid increases, which results in the necessity to increase the consumption of energy for pumping it out and for operation of the absorption column. Thus, the necessary effectiveness of operation of the installation and minimum energy consumption are ensured in the range of parameters indicated above.

In the method described above, a multistage process of reaction of the pumped vapor-gas medium, comprising hydrocarbons, with the hydrocarbon liquid is carried out. The first reaction takes place in a liquid-gas jet device in which the liquid medium consisting mainly of a hydrocarbon liquid provides for the pumping out and compression of the vapor-gas medium. A two-phase mixture is formed at the output from the jet device. During the reaction, the process of absorption of hydrocarbons from the vapor-gas medium by the liquid medium begins. The process continues up to the moment of separation of the mixture in the separator to a liquid medium and a gaseous phase, which is a partially hydrocarbon-removed and compressed vapor-gas medium.

Then the gaseous phase is fed to the absorption column where, as a result of reaction with the cooled hydrocarbon liquid (gasoline or kerosene fraction) fed therein, the process of reducing the content of hydrocarbons in the gaseous phase is carried out. Providing a counterflow system for movement of the gaseous phase and the hydrocarbon liquid in the absorption column creates more favorable conditions for absorption of hydrocarbons from the gaseous phase by the cooler hydrocarbon liquid. This makes it possible to significantly reduce the concentration of the hydrocarbons in the gaseous phase being cleaned as compared with their concentration in the vapor-gas medium. Feeding a portion of the liquid medium from the separator to an input of the pump makes it possible to create a circulation loop: separator—pump—liquid-gas jet device—separator, which reduces the consumption of fresh liquid medium fed to the installation from other sources. Feeding the hydrocarbon liquid from the absorption column to the separator or to the input of the pump provides for the creation of a process for renewing the liquid medium fed into the liquid-gas jet device. The process of renewing the circulating liquid medium may be carried out as a result of feeding petroleum products (gasoline or kerosene fraction of petroleum refining) to the separator or to the input of the pump. Since in the process of operation of the installation, hydrocarbons from the vapor-gas medium transfer into the liquid medium, it is advisable to remove it from the installation, for example, into the container for storage of petroleum products or into the tank to be filled.

As already noted, in the process of compression of the vapor-gas medium, the possibility is provided for carrying out absorption of hydrocarbons harmful for the environment. It should be noted that the process of uptake or, in other words, the process of absorption, which is understood to mean the process of dissolving gases in a liquid medium, makes it possible to reduce the consumption of energy in a liquid-gas jet device for the compression of a vapor-gas medium comprising hydrocarbons. This is achieved as a result of the fact that during the compression and transport of the vapor-gas mixture into the separator two independent processes are carried out—mechanical compression due to the kinetic energy of the jet of liquid medium and dissolution of a part of the vapor-gas mixture in the liquid medium, wherein the latter process is intensified as the pressure increases in the flowing part of the jet device and in the pipeline following it. The removal of the liquid medium from the loop of its circulation and the supply of fresh liquid medium into it provides the possibility to stabilize the makeup of the liquid medium fed into the jet device—a sorbent of hydrocarbon vapors. This provides more stable operation of the jet device and simultaneously maintains the absorption capability of the liquid medium. As a result it was possible to achieve balanced operation of the liquid-gas jet device and remove the hydrocarbon-removed gaseous phase from the absorption column into the environment without introducing contamination into the environment.

In a number of cases the absorption column cannot sufficiently effectively clean the gaseous phase fed from the separator of gaseous hydrocarbon impurities. This is related to the physical nature of the process of absorbing the gaseous hydrocarbons with the aid of a hydrocarbon liquid. No matter how strongly the hydrocarbon liquid is cooled, the content of the gaseous hydrocarbons in the gaseous phase cannot be reduced below the value of the partial pressure of the hydrocarbon liquid. Thus, by increasing the dimensions of the absorption column or making its construction more complex it is not possible to achieve reduction of the content of gaseous hydrocarbons in the gaseous phase below the value of the partial pressure of the hydrocarbon liquid at its operating temperature.

In the case where the gaseous phase comprises a large percent of hydrocarbons in its makeup, it is advisable to carry out subsequent cleaning with the use, for example, of a vortical tube or a gasdynamic separator for additionally cooling the gaseous phase and separating therefrom the condensate of hydrocarbons and other impurities.

It is also possible to use the installation at the output from the absorption column of the membrane device to remove from the gaseous phase the residues of gaseous hydrocarbons and other impurities, the molecules of which differ from the molecules of the main gases determining the chemical makeup of the air.

Use of the membrane device makes it possible to significantly reduce the ejection of substances harmful to the environment into the environment and to provide for the possibility, if that will be economically advisable, to supply gas enriched with hydrocarbons to the input into the installation for subsequent utilization. This may be achieved by pumping hydrocarbon-enriched gas out of the membrane device with the aid of the liquid-gas jet device. As a result the hydrocarbons are returned to the installation for repeated absorption, and the pressure drop increases in the membrane device, which intensifies the process of separating and cleaning the gaseous phase therein supplied from the absorption column. Further optimization of operation of the installation may be achieved by pumping hydrocarbon enriched gas from the membrane device with the aid of an additional liquid-gas jet device, which may provide the most favorable parameters for operation of the membrane device.

As a result, an effective method of removing hydrocarbons from a vapor-gas medium was created, which provides for a reduction of the loss of petroleum products and the exhaust of hydrocarbon-removed gas and other organic compounds into the environment without doing damage to the latter.

### BRIEF DESCRIPTION OF THE DRAWINGS

FIG. 1 is a schematic diagram of the installation in which a method is carried out for removing hydrocarbons from a vapor-gas medium formed during petroleum product storage and when filling a tank therewith.

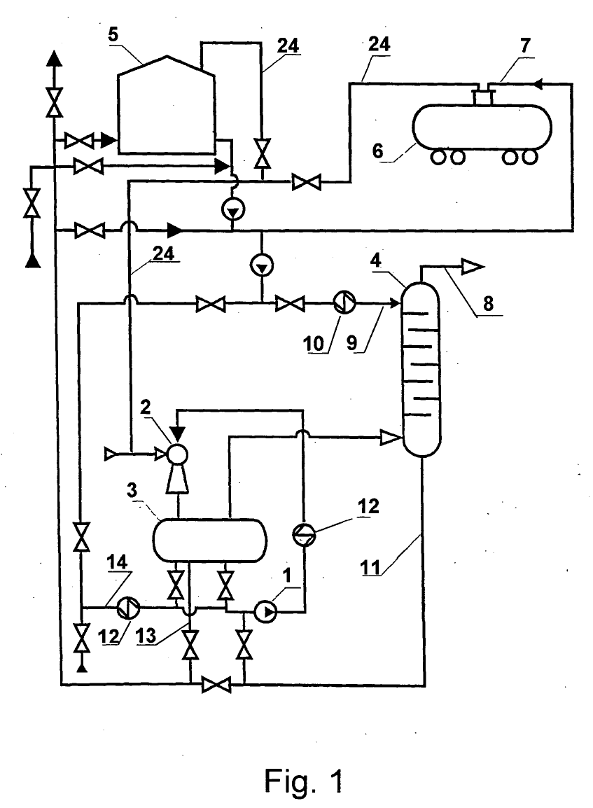

FIG. 2 is a variant of embodiment of the installation with a vortical tube.

FIG. 3 is a variant of embodiment of the installation with a gasdynamic separator.

FIG. 4 is a variant of embodiment of the installation with a membrane device.

### EMBODIMENTS OF CARRYING OUT THE INVENTION

The installation comprises a pump 1, a liquid-gas jet device 2, a separator 3 and an absorption column 4. The liquid-gas jet device 2 is connected by its liquid medium input to an output of the pump 1 and by a vapor-gas medium input by means of a pipeline 24 to a source of this medium —a tank with petroleum products, for example, a container 5 for storage of petroleum products or a tank 6, for example, a cistern filled with petroleum products (gasoline or kerosene), along a pipeline 7. The liquid-gas jet device 2 is connected from a mixture output to the separator 3. The liquid medium output of the separator 3 may be connected to an input into the pump 1. As a result of this, a loop for circulation of the liquid medium is formed, the circulation being sequential movement of the liquid medium from the pump 1 to the liquid-gas jet device 2, further from it to the separator 3 and from it to the input of the pump 1.

The absorption column 4 is connected from the side of the input therein of the gaseous phase to the output of the latter from the separator 3, wherein the absorption column 4 may be positioned above the level of the liquid medium in the separator 3. The upper part of the absorption column 4 is connected to a pipeline 8 for the output, for example, into the environment, of the gaseous phase clean of hydrocarbons, and to a pipeline 9 for supply of hydrocarbon liquid (gasoline or kerosene fraction) cooled by means of a refrigerator 10. In this variant of embodiment of the installation, the absorption column 4 is coupled at its lower part to the separator 3 by means of a pipeline 11. Wherein the removal of the hydrocarbon liquid with hydrocarbons dissolved therein is possible from the absorption column 4 not only to the separator 3, but also to other elements of the circulation loop of the liquid medium, for example, to the pump 1 from the side of the input therein of the liquid medium from the separator 3, and also to the container 5 for storage of petroleum products or to the tank 6.

The installation may be provided with a heat exchanger—refrigerator 12 for stabilization of the temperature of the liquid medium in the installation. The removal of the liquid medium from the separator 3 to the container 5 for storage of the petroleum products or to the tank 6 to be filled is carried out along a pipeline 13.

The installation is provided with a pipeline 14, along which the petroleum products (gasoline or kerosene) are supplied to the separator 3 or to the input of the pump 1.

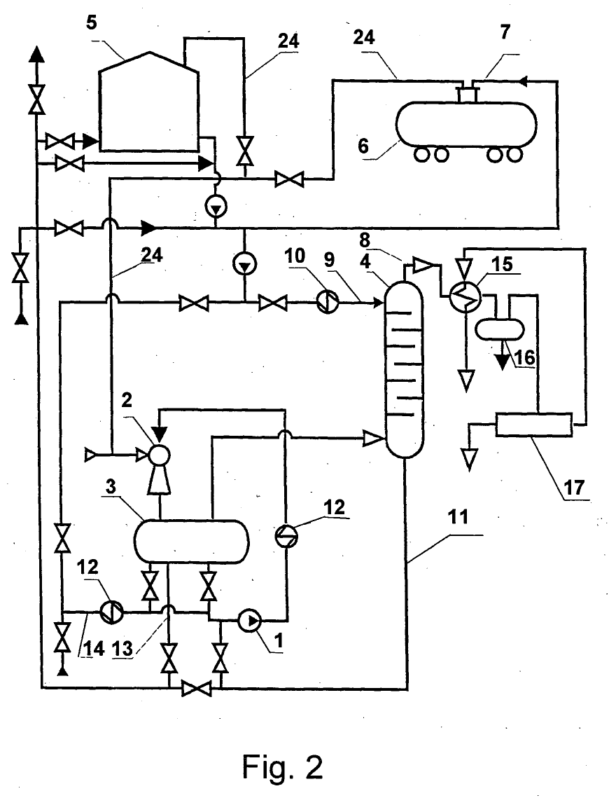

In FIG. 2 an installation is shown that is provided with an additional refrigerator-heat exchanger 15, an additional separator 16 and a vortical pipe 17. In the additional separator 16 a condensate formed during the cooling of the gaseous phase in the refrigerator-heat exchanger 15 is separated from the gaseous phase, and in the vortical pipe 17 the gaseous phase is separated into cold and warm gas mediums. The cold gas medium is directed to the additional refrigerator-heat exchanger 15 to cool the gaseous phase after the exit of the latter from the absorption column 4.

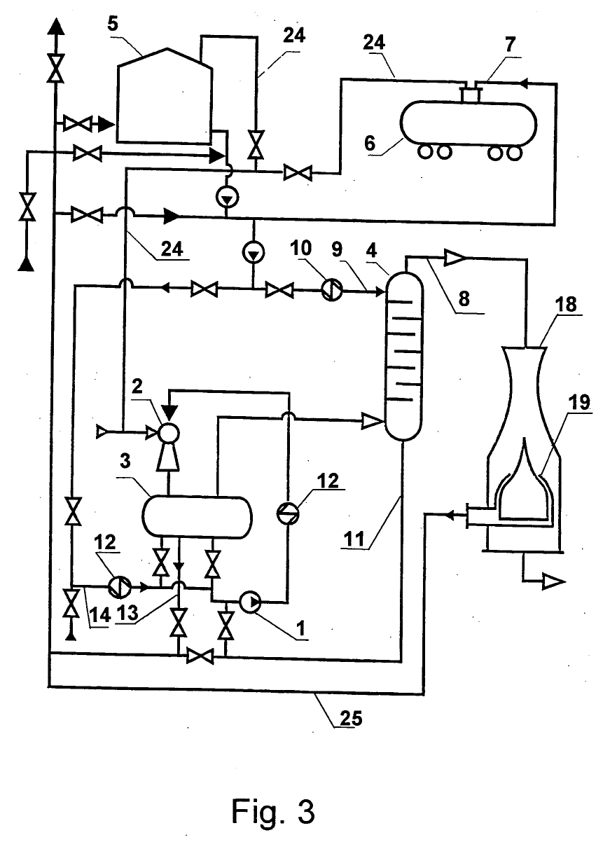

An installation is shown in FIG. 3, which is provided with a gasdynamic separator 18, serving to accelerate and expand the gaseous phase with the formation of vapors of hydrocarbons in the flow of condensate, then the condensate is separated from the gaseous phase in a dynamic separator 19 and removed from the gasdynamic separator 18 by means of a pipeline 25.

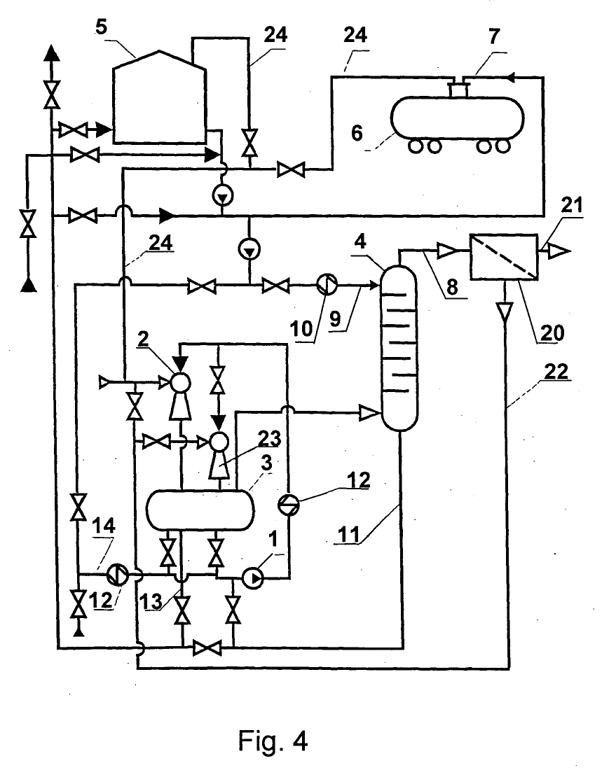

FIG. 4 shows an installation in which a membrane device 20 with pipelines 21 and 22 for the removal of, respectively, gas leaned of hydrocarbons and gas enriched by hydrocarbons may be connected to the pipeline 8 for the removal of the hydrocarbon-removed gaseous phase from the absorption column 4. The pipeline 22 may be connected to the liquid-gas jet device 2.

Furthermore, the installation may be provided with an additional liquid-gas jet device 23 connected by its liquid medium input to the output of the pump 1 and by its gaseous medium input to the pipeline 22 for the removal of hydrocarbon enriched gas. The output of the additional liquid-gas jet device 23 is connected to the separator 3.

During the storage of petroleum products in the container 5 and during the filling therewith of the tank 6 along the pipeline 7 a vapor-gas medium is formed, which is exhausted into the atmosphere. In order to organize the process of removing hydrocarbons from the vapor-gas medium, the liquid medium is fed by the pump 1 under pressure to the nozzle of the liquid-gas jet device 2 and the vapor-gas medium is pumped by the latter along the pipelines 24 from the tank 6 to be filled with petroleum products or from the container 5 for storage of petroleum products. In the liquid-gas jet device 2 the vapor-gas medium is compressed due to the energy of the liquid medium and is partially absorbed by the liquid. The mixture of vapor-gas and liquid mediums formed in the liquid-gas jet device 2 is fed therefrom into the separator 3. The mixture supplied to the separator 3 is separated into the gaseous phase and the liquid medium. A portion of the liquid medium may be fed from the separator 3 to the input of the pump 1, which provides for the creation of a loop for circulation of the liquid medium. Petroleum products (gasoline or kerosene fraction of petroleum refining) are fed to the separator 3 or to the input of the pump 1 along the pipeline 14. Simultaneously the excess liquid medium is removed from the separator 3 along the pipeline 13, for example, to the container 5 or to the tank 6 to be filled with petroleum products. In order to stabilize the temperature of the liquid medium fed to the liquid-gas jet device 2, refrigerators 12 may be installed in the loop for circulation of the liquid medium or on the pipeline 14.

An important parameter of the method for removing hydrocarbons from the vapor-gas medium is the value of compression of the vapor-gas medium in the liquid-gas jet device 2. It is advisable that the vapor-gas medium be compressed to a pressure in the separator 3, which is within the range of pressures from 0.20 MPa to 1.50 MPa. Compression of the vapor-gas medium in the liquid-gas jet device 2 below a pressure of 0.20 MPa makes it possible to reduce the consumption of electric energy for the operation of the pump 1 feeding the liquid medium to the jet device 2, but in that case the effectiveness of the process of absorption is reduced, which in turn results in an increase of the consumption of energy for cooling the hydrocarbon liquid fed to the absorption column 4. Compression of the vapor-gas mixture in the liquid-gas jet device 2 above the pressure of 1.5 MPa intensifies the process of absorption of hydrocarbons from the vapor-gas medium, but the consumption of electric energy for operation of the liquid-gas jet device 2 significantly increases, and this increase is not covered by the energy saved due to intensification of the process of absorption in the liquid-gas jet device 1 and in the absorption column 4.

It should be noted that the parameters of operation of the liquid-gas jet device 2 and the refrigerator 10 are interrelated. It was established during research that the necessary effectiveness of operation of the installation with minimum consumption of electric energy is achieved in the aforesaid range of the parameters.

The gaseous phase from the separator 3 is fed to the absorption column 4 into which a hydrocarbon liquid is fed as the absorbent. The process of absorption of the hydrocarbons from the gaseous phase by the hydrocarbon liquid is carried out in the absorption column 4, after which the hydrocarbon-removed gaseous phase and the hydrocarbon liquid with hydrocarbons dissolved therein are separately removed from the absorption column 4. A gasoline or kerosene fraction of petroleum refining is used as the hydrocarbon liquid. Prior to being fed to the absorption column 4, the hydrocarbon liquid is cooled with the aid of the refrigerator 10 to a temperature within the range of from minus 10° C. to minus 50° C.

Furthermore, the gaseous phase after output from the absorption column may be additionally cooled in an additional refrigerator—heat exchanger 15, and then in an additional separator 16 a condensate, which was formed as a result of cooling the gaseous phase, is separated therefrom. The gaseous phase is fed from the additional separator 16 to the vortical pipe 17 in which the gaseous phase is separated into cold and warm gas mediums. The warm gas medium is removed from the vortical pipe while the cold gas medium is fed to the additional refrigerator—heat exchanger 15 to cool the gaseous phase.

The gaseous phase from the absorption column 4 may be supplied to the gasdynamic separator 18 in which the gaseous phase as a result of its expansion and acceleration is cooled with the formation in the flow of a condensate from the hydrocarbon vapors remaining in the gaseous phase, then the condensate is separated from the gaseous phase in the dynamic separator 19 and removed from the gasdynamic separator 18 by means of the pipeline 25.

Furthermore, the gaseous phase may be fed from the absorption column 4 to the membrane device 20 in which the gaseous hydrocarbons remaining in that phase are separated therefrom, then the hydrocarbon leaned gas along the pipeline 21 and the hydrocarbon enriched gas along the pipeline 22 are separately removed from the membrane device 20. The hydrocarbon enriched gas may be pumped from the membrane device 20 by the liquid-gas jet device 2 or by the additional liquid-gas jet device 23.

The hydrocarbon liquid with the hydrocarbons dissolved therein may be fed from the absorption column 4 to the separator 3 or to the input of pump 1.

The hydrocarbon liquid with the hydrocarbons dissolved therein may be fed from the absorption column 4 to the container 5 for petroleum product storage or to the tank 6 to be filled therewith.

### INDUSTRIAL APPLICABILITY

The method for removing hydrocarbons from a vapor-gas medium formed during petroleum product storage and when filling a tank therewith may be used on gantries for filling and bases for storing petroleum products, and also in the chemical, petrochemical and other fields of industry.

1. A method for removing hydrocarbons from a vapor-gas medium formed during petroleum product storage and when filling a tank therewith, comprising pump supplying a liquid medium to a liquid-gas jet device, pumping out vapor-gas medium with this device from a tank filled with petroleum products or from a petroleum product storage container, compressing the vapor-gas medium in the liquid-gas jet device by energy of the liquid medium, feeding a mixture formed in the liquid-gas jet device of the vapor-gas and liquid mediums to a separator, separating the mixture in the separator into a gaseous phase and a liquid medium with removal of the gaseous phase and the liquid medium from the separator, wherein the gaseous phase is fed from the separator to an absorption column into which a hydrocarbon liquid is fed as an absorbent, the process of absorption of hydrocarbons from the gaseous phase is carried out by the hydrocarbon liquid in the absorption column, then the hydrocarbon-removed gaseous phase and the hydrocarbon liquid containing hydrocarbons of the gaseous phase dissolved therein are separately evacuated from the absorption column, characterized in that a gasoline or kerosene fraction of petroleum refining is used as the hydrocarbon liquid, and prior to being fed to the absorption column the hydrocarbon liquid is cooled to a temperature within the range of from minus 10° C. to minus 50° C., and the pressure of the mixture of the vapor-gas and liquid mediums, which is formed in the liquid-gas jet device, is maintained in the separator at a level ranging from 0.2 to 1.5 MPa.

2. The method according to claim 1, characterized in that a portion of the liquid medium from the separator is fed to a pump input.

3. The method according to claim 1, characterized in that the gaseous phase after exit from the absorption column is additionally cooled, wherein a condensate formed as a result of cooling the gaseous phase is separated therefrom in an additional separator, and the gaseous phase is fed from the additional separator to a vortical pipe in which the gaseous phase is separated into cold and warm gas mediums, then the warm gas medium is removed into the atmosphere and the cold gas medium is used to cool the gaseous phase after the exit of the latter from the absorption column.

4. The method according to claim 1, characterized in that the gaseous phase from the absorption column is fed to a gasdynamic separator in which the gaseous phase as a result of its acceleration and expansion is cooled with the formation in the stream of a condensate from the hydrocarbon vapors remaining in the gaseous phase, then the condensate is separated from the gaseous phase and removed from the gasdynamic separator.

5. The method according to claim 1, characterized in that the hydrocarbon liquid with the hydrocarbons of the gaseous phase dissolved therein is fed from the absorption column to a separator or to a pump input.

6. The method according to claim 1, characterized in that the hydrocarbon liquid with the hydrocarbons of the gaseous phase dissolved therein is fed from the absorption column into a container for storing petroleum products or into a tank to be filled therewith.

7. The method according to claim 1, characterized in that the liquid medium is removed from the separator into a container for storing petroleum products or into a tank to be filled therewith.

8. The method according to claim 1, characterized in that petroleum products, a gasoline or kerosene fraction of petroleum refining, are fed to the separator or to the pump input.

9. The method according to claim 1, characterized in that the gaseous phase after exit from the absorption column is fed to a membrane device in which the gaseous hydrocarbons remaining therein are separated therefrom, then gas depleted of hydrocarbons and hydrocarbon-enriched gas are separately removed from the membrane device.

10. The method according to claim 9, characterized in that the hydrocarbon-enriched gas is pumped from the membrane device by the liquid-gas jet device.

11. The method according to claim 9, characterized in that the hydrocarbon-enriched gas is pumped from the membrane device by an additional liquid-gas jet device.
